(19) United States
(12) Patent Application Publication
(10) Pub. No.: US 2024/0350724 A1
(43) Pub. Date: Oct. 24, 2024

(54) DEVICE FOR CONTROLLED DISPENSING OF FLUID

(71) Applicant: Master Shreyan Ninad PHADKE, Santa Clara, CA (US)

(72) Inventors: Anand Vasant BAM, Pune (IN); Mrinalini Anand BAM, Pune (IN)

(21) Appl. No.: 18/686,420

(22) PCT Filed: Aug. 27, 2022
(86) PCT No.: PCT/IN2022/050763

§ 371 Date: Feb. 25, 2024

(30) Foreign Application Priority Data
Aug. 27, 2021 (IN) 202121026221

(51) Int. Cl. A61M 5/142

(52) U.S. Cl. CPC A61M 5/14216

(57) ABSTRACT

A device for controlled dispensing of fluid 100 is described. The device for controlled dispensing of f) dispenses fluid in micro quantities within predefined amount of time in a dispensing cycle. A primary tank 105 including liquid is positioned preferably over a plunger unit 120. A controller 115 configured to dispense fluid in dispensing cycles for repeatedly dispensing the fluid by activating an electromagnet 205 of the plunger unit 220. A plunger 215 of the plunger unit 120 reciprocally movable from a first position to a second position and vice versa to release fluid to a needle unit 125. The removable and replaceable needle unit 125 transfers predefined quantity of the fluid received from a tube 110 to a vial 130.

### FIELD OF THE INVENTION:

The present invention relates to fluid dispensing devices and more particularly relates to precisely controlled fluid dispensing devices.

### BACKGROUND OF THE INVENTION:

Precision and accuracy are important factors in the manufacturing industry. In addition to these parameters repeatability is also a parameter that is considered in the manufacturing industry. It is important to have accurate products or deliveries with repeatability. It is observed that dispensing small quantities of liquids with accuracy and repeatability is challenging.

In chemical, pharma industry, and in the various fields of research, dispensing equal dosage of fluids repetitively is very crucial as the over or under dosage may affect the subject to an exceptionally large extent. The accuracy and repeatability are of utmost important in such cases. Currently, the systems for creating equal dosages of the medicines/liquids, injections or any other fluids are mostly automated. Such systems use devices for dispensing predefined quantity of fluids. The accuracy of the automated devices and processes is far greater than that of the human.

U.S. Pat. No. 5,312,233A to IVEK CORP is a linear liquid dispensing pump for dispensing liquid in nanolitre volumes. The U.S Patent discloses a dispensing pump that is capable of dispensing volumes in nanolitre with an accuracy of 0.1% or better. The linear liquid dispensing pump utilizes digital electronics, solid-state sensors and integrates readily with a microprocessor or like control with close loop feedback electronic circuit.

Korean Patent KR100518145B1 to Musashi Engineering Inc. discloses a plunger for a syringe of liquid dispenser. The Korean Patent has a liquid dispenser that discharges a small amount of liquid from a needle connected to a tip of a syringe by pressing a liquid material such as an adhesive or resin filled in a syringe with compressed air.

There is a need of device for dispensing precise amounts of fluids. There is also need of device for precise and controlled dispensing of fluids repeatedly.

### SUMMARY OF THE INVENTION:

A device for controlled dispensing of fluid for dispensing predefined quantity of liquid with accuracy, precision and repeatability is disclosed. The device of the present invention (hereinafter referred to as “the device”) includes a primary tank, a tube, a controller, and a plunger unit including a plunger that has at least two positions such as a first position and a second position.

The primary tank stores fluid that is flowable from the tube by the force of gravity. The primary tank is positioned over the plunger unit. The tube passes through a bottom end of the plunger unit while receiving fluid from the primary tank. The controller is configured to dispense fluid in dispensing cycles for repeatedly dispensing the fluid by activating the electromagnet of the plunger unit. The needle unit is removable and replaceable. The needle unit transfers predefined quantity of the fluid received from the tube to the vials in one or more dispensing cycles.

The plunger of the plunger unit is reciprocally movable from the first position to the second position and vice versa. In the first position of the plunger, the flow of liquid in the tube is blocked. In the second position of the plunger, the flow of liquid in the tube is released advantageously to the needle unit. The plunger unit includes an electromagnet, the plunger and a spring, a flange, a tip, and a plate. The plunger unit is reciprocally movable by the electromagnet that has the spring that is positioned on the plunger to position the plunger in the first position. The tube passes through a bottom end of the plunger unit.

The primary tank is advantageously sealed and pressurised to defining a non-free flow fluid flow through the tube. A first end of the tube is connected to a bottom portion of the primary tank, and a second end of the tube is connected to the needle unit. The tube is positioned by passing through the plunger unit. The tube is guided by tube guides while entering the plunger unit and also while exiting the plunger unit. The tube is elastic, chemical resistant and resilient material. The fluid flows through the tube due to gravitational force in the second position of the plunger unit. The controller is defined as a timer. The tip of the plunger unit presses a portion of the tube being positioned immediately below the tip. The position of the tank is changeable in accordance with the present invention.

### BRIEF DESCRIPTION OF THE DRAWINGS:

The objectives and advantages of the present invention will become apparent from the following description read in accordance with the accompanying drawings wherein

FIG. 1 is a front view of a device for controlled dispensing of fluid in accordance with a preferred embodiment of the present invention;

FIG. 2 is an enlarged front view of a plunger unit of the device for controlled dispensing of fluid of FIG. 1;

FIG. 2A shows an exploded view of the device for controlled dispensing of fluid of FIG. 1;

FIG. 3 shows a first position of a plunger of the plunger unit of FIG. 1;

FIG. 3A shows a second position of a plunger of the plunger unit of FIG. 1;

FIG. 3B shows flowchart showing steps in the operation of the controller the device for controlled dispensing of fluid of FIG. 1;

FIG. 3C is a graph showing liquid dispensed at different time durations;

FIG. 3D is a graph showing liquid dispensed by various nozzles at different time durations;

FIG. 4 shows a first embodiment of the device for controlled discharge of fluids 100 of FIG. 1;

FIG. 5 shows a second embodiment of the device for controlled discharge of fluids 100 of FIG. 1; and

FIG. 6 shows a third embodiment of the device for controlled discharge of fluids 100 of FIG. 1.

### DETAILED DESCRIPTION OF THE DRAWINGS:

The invention herein is described using specific exemplary details for better understanding. However, the invention disclosed can be worked on by a person skilled in the art without the use of these specific details.

References in the specification to “one embodiment” or “an embodiment” means that particular feature, structure, characteristic, or function described in connection with the embodiment is included in at least one embodiment of the invention. The appearances of the phrase “in one embodiment” in various places in the specifications are not necessarily all referring to the same embodiment.

References in the specification to “preferred embodiment” means that a particular feature, structure, characteristic, or function described in detail thereby omitting known constructions and functions for clear description of the present invention.

The present invention is illustrated with reference to the accompanying drawings, throughout which reference numbers indicate corresponding parts in the various figures.

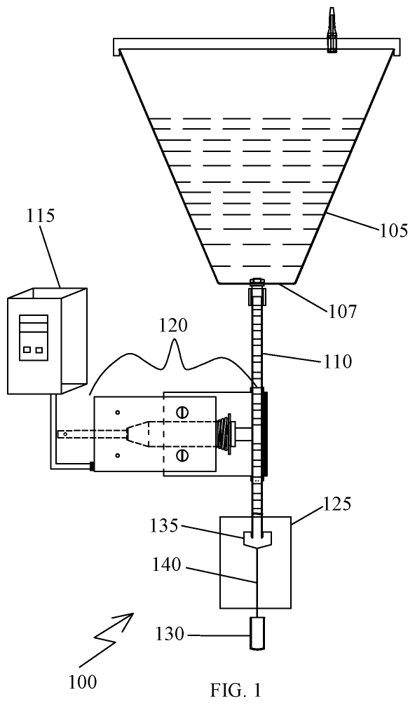

Referring to FIG. 1, a device for controlled dispensing of fluid 100 is described. The device for controlled dispensing of fluid 100 (hereinafter referred to as “the device”) includes a primary tank 105, a tube 110, a controller 115, a plunger unit 120, a needle unit 125 and a vial 130. The primary tank 105 is positioned preferably over the plunger unit 120 in accordance with the present invention.

The primary tank 105 stores fluid, for example, water, oil or free-flow/non-free-flow solutions or the like. The fluid stored in the primary tank 105 is flowable from the tube 110 due to gravity. It is however, noted that in case of non-free flow fluids, the primary tank 105 is sealed and pressurised in accordance with methods known in the prior art to make the non-free flow fluid flow through the tube 110.

A first end of the tube 110 is connected to a bottom portion 107 of the primary tank 105 and a second end of the tube 110 is connected to the needle unit 125. The tube 110 advantageously passes through the plunger unit 120 in accordance with the present invention. The tube 110 is guided by tube guides while entering the plunger unit 120 and also while exiting the plunger unit 120.

In the present embodiment, the tube 110 is elastic in nature and preferably made of resilient and relatively chemically-resistant plastics or rubber, such as Teflon tubing, silicone tubing and the like. It is however noted that, the tube 110 is made of other materials known in the art in the other embodiments of the present invention. In accordance with the present invention, fluid flows through the tube 110 due to gravitational force.

The needle unit 125 includes an adapter 135 and a needle 140. One end of the needle 140 is received in the adapter 135 and the other end is received in the vial 130. The needle unit 125 is removable and replaceable by approximately identical needle unit having different needle. The needle unit 125 is selected in accordance with the requirement of the dispensing requirement in the vial 130. The needle 125 transfers fluid received from the tube 110 to the vial 130.

In the present embodiment, the controller 115 that is connected with the plunger unit 120 controls operation the plunger unit 120. In other embodiments, the controller 115 is defined by microprocessor, programmable logic controller (PLC). The controller 115 is controls the plunger unit 120. The controller 115 preferably configured to operate the plunger unit for dispensing liquid in accordance with the timer set by a user. In an embodiment, the controller is defined by a timer or a timer circuit.

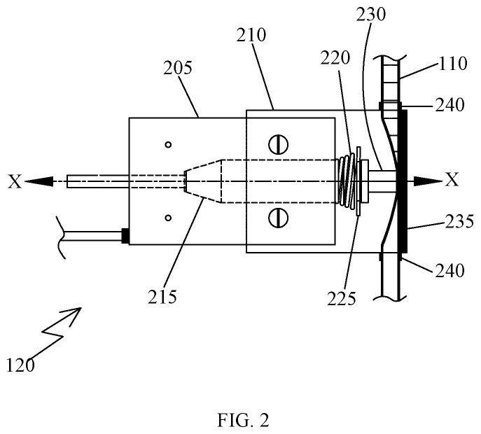

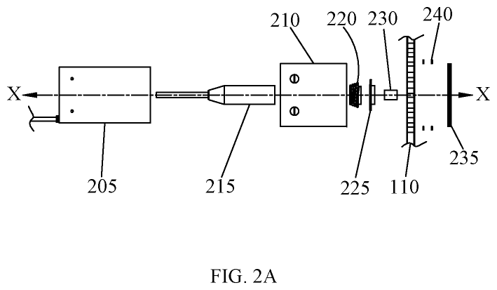

Now referring to FIGS. 1, 2 and 2A, the plunger unit 120 of the device 100 is described. The plunger unit 120 includes an electromagnet 205, a bracket 210, a plunger 215, a spring 220, a flange 225, a tip 230, and a plate 235. The tube 110 is receivable in a bottom end portion of the plunger unit 120. The bracket 210 advantageously securely holds electromagnet 205.

The plunger unit 120 also includes an opposed pair of tube guides 240. The first tube guide 240 is on a first end from which the tube 110 enters the plunger unit 120. The second tube guide 240 is on the second end from which the tube 110 exits from the plunger unit 120. The tip 230 is positioned by the effect of the spring 220 such that in a normal condition a portion of the tube 110 immediately below the tip 230 is pressed by the tip 230.

In accordance with the present invention, the electromagnet 205 is fixed with the bracket 210 of the device 100. The plunger 215 is reciprocally movable along axis-X in the electromagnet 205. The spring 220 is positioned on the plunger 215 such that the spring 220 is positioned between the flange 225 and the bottom end portion of the plunger 215.

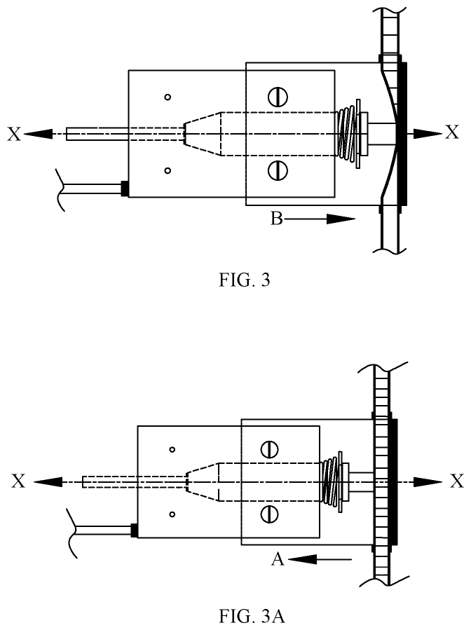

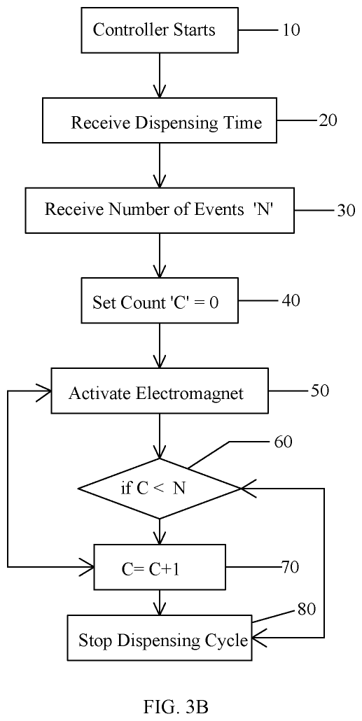

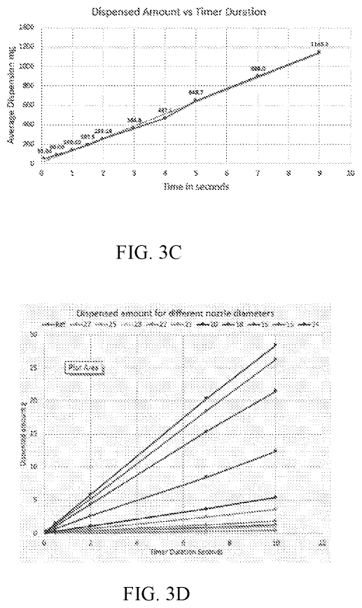

Referring to FIGS. 3-3A, a first position A and a second position B of the plunger 215 of the plunger unit 120 for controlled dispensing of fluids 100 are shown. The plunger 215 is in the first position A and that is displaced to the second position B by the controller 115 by activating the electromagnet 205. To achieve the second position B, the tip 230 of the plunger 215 moves in a direction indicated by arrow 'C. The movement is achieved by activating the electromagnet 205 that pulls the plunger 215 and the tip 230 releasing predefined pressure on the tube 110.

In order to move the plunger 215 from the first position A to the second position B, the controller 115 activates the electromagnet 205 such that the electromagnet 205 produces a force against the spring 225. Accordingly, the plunger 215 and the tip 230 are retracted by this force releasing the predefined force on the tube 110 applied by the tip 230. The plunger 215 and the tip 230 are pulled towards the direction indicated by arrow “C”.

Now, in the second position B of the plunger 215, the shape of the pressed portion of the tube 110 is restored. It is understood however that even after frequent operation of the plunger 215 the tube maintains the elastic property. When the plunger 215 is reciprocated from the first position A to the second position B, the pressed portion of the tube 110 is released and predefined quantity of liquid flows through the tube 110 to the needle unit 125. The fluid is dispensed from the needle 140 till the plunger 215 is in the second position B.

After the required amount of fluid is dispensed, the plunger 215 is reciprocated back to the first position A in the direction indicated by arrow-D. The controller 115 stops sending signals and current to the electromagnet 205 and the spring 220 returns to its original position. As the first position A of the plunger 215 is achieved, the tip 230 presses the portion of the tube 110 that is exactly below the tip 230 and flow of the fluid from the tube 110 is blocked ultimately no fluid is dispensed from the needle 140 in the vial 130.

As the fluid is dispensed out and the vials 130 are filled, the level of the fluid in the primary tank 105 reduces marginally. It is understood that to maintain precision in dispensed volumes of fluid, it is necessary to maintain constant level of fluid in the primary tank 105.

FIG. 3B shows a flow chart that shows steps involved in an operation of the controller to dispense fluid in accordance with the present invention. In a first step 10, the controller is activated. In a second step 20, the user gives dispensing time as input to the controller, for example, time in seconds or microseconds. The input is provided by the use to the controller by any input means like a keyboard or a touch screen or the like. In a next step 30, the count for the cycle is entered by the user. For3 example, if the count is 5 then the controller performs 5 events of dispensing the liquid in respective vials for given amount of time as per the second step. One dispensing cycle may involve one or more events of dispensing liquid in the respective vials. In a next step 40, the controller sets a reference count C to zero.

In a next step 50, the controller activates the electromagnet 205 for predefined amount of time as per the user input in the second step. In this step, liquid is dispensed in the vial till the electromagnet 205 is activated i.e. the plunger 215 is in the second position. In next steps 60 and 70, the dispensing counter is checked and accordingly the control goes to the previous step till the count of the number of dispensing events is achieved. In a next step 80, the electromagnet6 205 is deactivated after and the dispensing cycle is finished.

Now referring to FIGS. 1-3B, the operational flow of the device for controlled dispensing of fluid 100 is described. In operation, the user of the device 100 maintains the appropriate level of fluid in the primary tank 105 of the device 100. The device 100 ideally is resting in the first position of the plunger. In the first position, the controller 115 does not supply electricity and electromagnet does not signal signals to the electromagnet 205. The user gives input of the time of dispensing and the number of events of dispensing in a dispensing cycle.

In the first position A, the tension on the spring 220 is released such that the tip 230 of the plunger 215 is pushed towards the guide 225 along the X-Axis. Simultaneously, in the first position A the plunger 215 and the tip 230 are pulled towards the tube 110 along with the tip 230 along the length of X-Axis by spring force. The tip 230 of the plunger 215 pushes the tube 110 between the two tube guides 240 towards the plate 235 to block the flow of fluid from the tube 110. The plate 235 withstands the force exerted through the plunger 215 to block the flow of fluid through the tube 110.

The device 100 dispenses the fluid by moving the plunger 215 from the first position A to the second position B of the plunger 215. In the first position A, the controller 115 activates the electromagnet 205 where the electromagnet 205 induces electric current. The electromagnet 205 pulls the tip 230 of the plunger 215 creating tension in the spring 220 such that the tip 230 of the plunger 215 is pulled in the direction indicated by arrow-C.

The movement of the plunger 215 from a first position A to the second position B releases the fluid inside the tube 110. In the second position B, the pressed portion of the tube 110 is released and predefined quantity of liquid flows through the tube 110 to the needle unit 125. The fluid is dispensed from the needle 140 till the plunger 215 is in the second position B.

After the required amount of fluid is dispensed, the controller 115 stops the signals to the electromagnet 205. The spring 220 is recoiled and the tip 230 of the plunger 215 presses the portion of the tube 110 below the tip 230. The plunger 215 returns to the first position A and flow of the fluid from the tube 110 is blocked ultimately no fluid is dispensed from the needle 140 in the vial 130.

In a preferred embodiment of the present invention, the amount of fluid dispensed by the device 100 depends on the amount of time the plunger 215 is in the second position B. Accordingly, the controller 115 controls and actuates the electromagnet 205 to control the amount of dispensed fluid in accordance with inputs received from the user.

For example, dispensing cycle that was performed using the device for controlled dispensing of fluid 100 of the present invention. Following results were found with the preferred embodiment of the present invention. Accordingly, the controller was configured to set a timer of capacity 10 seconds with least count of 0.01 seconds to set the time for opening the tube 110 to release liquid to the needle unit 125 that reaches to the vial 130. A weigh balance of 50 g capacity having least count of 0.01 g was used to weigh the dispensed volume out of the needle unit 125. In this example, two types of centrifuge tubes of 100 μliter and 2 ml capacity were used to collect the dispensed fluid. Single needle was used and set of 30 readings were noted. Following observations were made. (Refer Table 1 and FIG. 3C).

TABLE 1
Dispensed amount at various time durations

| | | Timer Duration (Seconds) | | | | | | | | | | |
|---|---|---|---|---|---|---|---|---|---|---|---|---|
| | | 0.1 | 0.5 | 1.0 | 1.5 | 2.0 | 3.0 | 4.0 | 5.0 | 7.0 | 9.0 | 15.0 |
| | | Average dispensed amount (mg) | | | | | | | | | | |
| | | 51.9 | 90.0 | 140.6 | 193.5 | 255.2 | 364.8 | 467.1 | 648.7 | 899.0 | 1145.8 | 1877.1 |
| | | Standard Deviation | | | | | | | | | | |
| | | 5.9 | 13.2 | 12.7 | 19.4 | 11.2 | 9.6 | 10.7 | 11.8 | 11.6 | 9.6 | 18.5 |
| Range | Max Reading | 66.7 | 110.0 | 160.0 | 220.0 | 270.0 | 380.0 | 480.0 | 670.0 | 930.0 | 1160.0 | 1900.0 |
| | Min Reading | 40.0 | 70.0 | 120.0 | 160.0 | 220.0 | 350.0 | 450.0 | 630.0 | 880.0 | 1130.0 | 1830.0 |

In another cycle of operation, various needles were used. Accordingly, five different readings of dispensed fluid were noted using different time settings. The readings were tabulated, and graphs were drawn.

Dispensed amount of fluid at various timer durations using 10 different nozzle diameters were measured (Refer FIG. 3D). It was observed that repeat accuracy was maintained for 5 replicates. It was also observed that a linear relationship between timer duration and amount dispensed was maintained for all nozzle diameters.

In another embodiment of the present invention, the plunger unit 120 of the device 100 is replaceable by other reciprocating units such as pneumatic cylinder, hydraulic cylinder, Eccentric mechanical device etc.

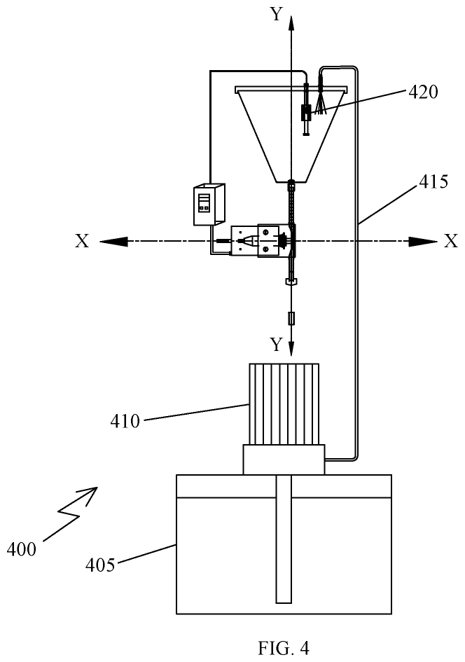

As shown in FIG. 4, the first embodiment 400 of the device for controlled discharge of fluids 100 is described hereinafter. In this one embodiment, the device 400 has a tank 405, a motor 410, a pipe 415 and a float 420. The tank 405 has larger storage capacity of fluid relative to the primary tank 105. The tank 405 is positioned below the motor 410 along the X-Axis. The tank 405 is connected with the motor 410.

In this present embodiment, the motor 410 is preferably a pump motor however, the type of motor may vary in other embodiments of the present invention. The motor 410 is connected with the primary tank 105 through the pipe 415. The float 420 is positioned inside the primary tank 105 at a predefined position and connected with the controller 115.

The float 420 is configured to sense the level of the fluid inside the primary tank 105. The float 420 signals the controller 115 if the fluid level in the primary tank 105 is below the predefined level. The controller 115 upon receiving the signal from float 420 activates the motor 410. The motor 410 pumps the fluid from the tank 405 in the primary tank 105 through the pipe 415 maintaining the predefined fluid level in the primary tank 105. The float 420 signals the controller 115 once the fluid level is at the predefined level in the primary tank 105. Further, the controller 115 signals the motor 410 to stop the flow of fluid.

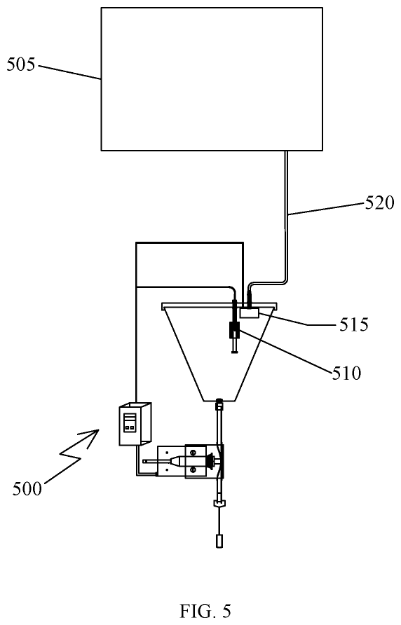

As shown in FIG. 5, the second embodiment 500 of the device for controlled discharge of fluids 100 is described hereinafter. In this one embodiment, the device 500 includes a second tank 505, a float 510, a valve 515 and a second pipe 520. The second tank 505 is advantageously positioned over the primary tank 105 along the Y-Axis. The valve 515 is operated through a prime mover for allowing and blocking the flow of fluid through the second pipe 520.

The second pipe 520 is connected with the base of the second tank 505 from one end and is connected with the valve 515 from another end. The float 510 is connected with the controller 115 preferably through a wired medium. The float 510 has a circular disc magnet (not shown) that glides along the length of the float 510 on Y-Axis. The controller 115 is also connected with the valve 515. The float 510 senses the level of fluid in the primary tank 105 and signals the controller 115.

When the level of fluid is below predefined fluid level, the float 510 signals the controller 115 accordingly. Further, the controller 115 opens the valve 515 for channelizing the fluid inside the primary tank 105 through the second pipe 520. The float 510 senses the level of the fluid and if the level of the fluid is up to the predefined fluid level, then the controller 115 is notified. The controller 115 closes the valve 515 stopping the flow of fluid in the primary tank 105.

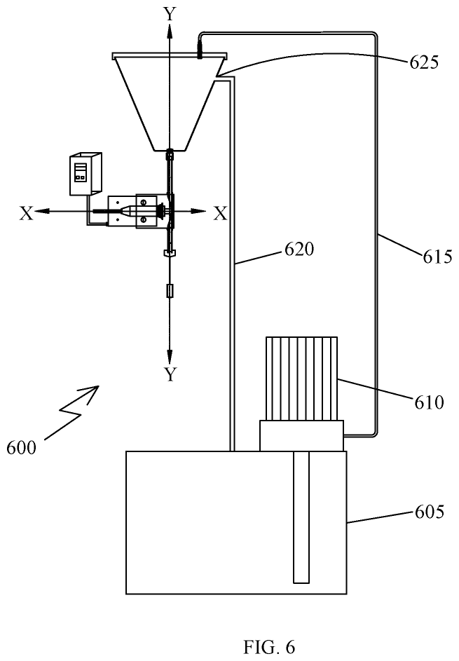

As shown in FIG. 6, the third embodiment 600 of the device for controlled discharge of fluids 100 is described. In this embodiment, the device 600 includes a third tank 605, a motor 610, a third pipe 615 and a fourth pipe 620. The third tank 605 is positioned below the plunger unit 120. The motor 610 is positioned on the third tank 605. The motor 610 is preferably a pump motor, however, the type of motor may vary in other embodiments of the present invention. The diameter of the fourth pipe 620 is twice as that of the third pipe 615.

The motor 610 is connected with the controller 115 through a wired medium. The third pipe 615 is connected with the motor 610 from one end and the primary tank 105 from another end. In this present embodiment the primary tank 105 has a slit 625 at a predefined position. The fourth pipe 620 is connected to the slit 625 from one end and to the third tank 605 from another end.

The controller 115 activates the motor 610 to pump the fluid from the third tank 605 towards the primary tank 105 through the third pipe 615. The motor 610 continuously pumps the fluid from the third tank 605 in the primary tank 105. The fluid in the primary tank 105 is filled up to the slit 625 and the excess fluid is drained from the slit through the fourth pipe 620 back to the third tank 605.

The device for controlled dispensing of the fluid 100 advantageously dispenses the fluid in multiples of nano to millilitres/multi litres with proper design of the various components. The device for controlled discharge of fluids 100 advantageously dispenses the accurate amount of fluid as per the user requirement as set by the user. The accuracy of quantity of fluid dispensed from the needle 140 is advantageously maintained even after frequent contraction and/or expansion of the tube 110. The device 100 maintains the repeatability of dispensing liquid in the vials 135 in addition to accuracy and precision.

The foregoing description of specific embodiments of the present invention has been presented for purposes of illustration and description. They are not intended to be exhaustive or to limit the present invention to the precise forms disclosed, and obviously many modifications and variations are possible in light of the above teaching.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the scope of the present invention.

1. A device for controlled dispensing of fluid 100 for dispensing predefined quantity of liquid repeatedly comprising:
a primary tank 105, the primary tank 105 including stored fluid being flowable from a tube 110, the primary tank 105 including fluid being positioned over a plunger unit 120;
the tube 110, the tube 110 passing through a bottom end of the plunger unit 120 receiving fluid from the primary tank 105;
a controller 115 configured to dispense fluid in dispensing cycles for repeatedly dispensing the fluid by activating an electromagnet 205 of the plunger unit 120;
a removable and replaceable needle unit 125 transferring predefined quantity of the fluid received from the tube 110 to a vial 130;
and a plunger 215 of the plunger unit 120 reciprocally movable from a first position to a second position and vice versa wherein the flow of liquid in the tube 110 is being blocked in the first position, and the flow is being released to the needle unit 125 in the second position.

2. The device for controlled dispensing of fluid 100 as claimed in claim 1, wherein the plunger unit 120 including the electromagnet 205, the plunger 215 and a spring 220, a flange 225, a tip 230, and a plate 235.

3. The device for controlled dispensing of fluid 100 as claimed in claim 1, wherein the plunger unit 120 being reciprocally movable by the electromagnet 205 including the spring 220 being positioned on the plunger 215 to position the plunger 215 in the first position.

4. The device for controlled dispensing of fluid 100 as claimed in claim 1, wherein the primary tank 105 being sealed and pressurised defining a non-free flow fluid flow through the tube 110.

5. The device for controlled dispensing of fluid 100 as claimed in claim 1, wherein a first end of the tube 110 being connected to a bottom portion 107 of the primary tank 105, and a second end of the tube 110 being connected to the needle unit 125.

6. The device for controlled dispensing of fluid 100 as claimed in claim 1, wherein the tube 110 being positioned by passing through the plunger unit 120

7. The device for controlled dispensing of fluid 100 as claimed in claim 1, wherein the tube 110 being guided by tube guides while entering the plunger unit 120 and also while exiting the plunger unit 120. 8 The device for controlled dispensing of fluid 100 as claimed in claim 1, wherein the tube 110 is elastic, chemical resistant and resilient material.

9. The device for controlled dispensing of fluid 100 as claimed in claim 1, wherein the fluid flows through the tube 110 due to gravitational force in the second position of the plunger unit 120.

10. The device for controlled dispensing of fluid 100 as claimed in claim 1, wherein the controller 115 being defined as a timer.

11. The device for controlled dispensing of fluid 100 as claimed in claim 1, wherein the tip 230 of the plunger unit 120 pressing a portion of the tube 110 being positioned immediately below the tip 230.

12. The device for controlled dispensing of fluid 100 as claimed in claim 1, wherein position of the tank 105 is changeable.
